(19) United States
(12) Patent Application Publication
(10) Pub. No.: US 2026/0083815 A1
(43) Pub. Date: Mar. 26, 2026

(54) COMPOSITION FOR INHIBITING TOXICITY OF NANOPARTICLES AND ENVIRONMENTALLY-DERIVED FINE PARTICLES

(71) Applicant: AJOU UNIVERSITY INDUSTRY-ACADEMIC COOPERATION FOUNDATION, Suwon-si (KR)

(72) Inventors: Gwang LEE, Suwon-si (KR); Tae-Hwan SHIN, Daegu (KR); Yup KANG, Yongin-si (KR); Jong-Young KWAK, Suwon-si (KR); Dayeon LEE, Ulsan (KR)

(73) Assignee: AJOU UNIVERSITY INDUSTRY-ACADEMIC COOPERATION FOUNDATION, Suwon-si (KR)

(21) Appl. No.: 19/360,290

(22) Filed: Oct. 16, 2025

Related U.S. Application Data
(63) Continuation of application No. 17/635,127.

(30) Foreign Application Priority Data
Aug. 14, 2019 (KR) 10-2019-0099363
Jul. 29, 2020 (KR) 10-2020-0094306

(51) Int. Cl. A61K 38/06; A61K 31/191

(52) U.S. Cl. CPC A61K 38/063; A61K 31/191

(57) ABSTRACT

The present invention relates to a composition for inhibiting the toxicity with respect to nanoparticles and particulate matters generated from the environment. Since it has been confirmed that a decrease in intracellular ATP, a decrease in cell viability, inflammation-induced morphological changes in cells and cell activation, which are induced by nanoparticles or environmentally-derived particulate matters, are inhibited by means of the composition of the present invention, the composition may be utilized as a reducing substance for the toxicity of nanoparticles and particulate matters.

### CROSS REFERENCE TO RELATED APPLICATIONS

This application is a continuation of U.S. patent application Ser. No. 17/635,127 filed on Feb. 14, 2022, which is a U.S. National Stage Application of International Application No. PCT/KR2020/010687, filed on Aug. 12, 2020, which claims the benefit under 35 USC 119(a) and 365(b) of Korean Patent Application No. 10-2019-0099363, filed on Aug. 14, 2019 and Korean Patent Application No. 10-2020-0094306, filed on Jul. 29, 2020, in the Korean Intellectual Property Office, the entire disclosure of which is incorporated herein by reference for all purposes.

### TECHNICAL FIELD

The present disclosure relates to a composition for inhibiting toxicity of nanoparticles and environmentally-derived particulate matters.

### BACKGROUND ART

Recently, technologies for nanoparticles have grown rapidly and are being applied to various industry fields such as manufacturing industry, medicine, food, and cosmetics. In addition, particulate matters such as black carbon, fine dust, and microplastics are generated through combustion and physical decomposition in the environment and may exist in air and water. However, as the scope of application of nanoparticles increases and the generation of environmentally-derived particulate matters increases, the exposure route of nanoparticles and particulate matters to the human body may be diversified, and the exposure frequency may also increase (Angew. Chem. Int Ed Engi, 2011, Arch Toxicol, 2017).

Accordingly, there is a need for toxicity-reducing materials for nanoparticles and particulate matters worldwide.

In general, nanoparticles refer to particles with an average diameter in the range of 1-100 nm, which has much greater specific surface area per volume than matters having large particles. Accordingly, reactivity occurring on a surface is quite high while having unique physical and chemical properties. Although these unique properties are industrially useful, potential toxicity may be induced in a safety standpoint.

In addition, it was reported that the smaller the size of the nanoparticles, the more harmful, and reactions such as cell death and inflammation were induced due to the generation of reactive oxygen species (J. Control Release, 2013). In addition, the degree of toxicity varies depending on a material forming the nanoparticles, and even the same material with the same size can have different toxicity depending on a structure, shape, and environment (Environ Health Perspect, 2006).

Inhalation, ingestion, and skin are known as main routes of inflow of nanoparticles, and nanoparticles introduced into the human body are distributed to all organs and may cause diseases in each organ (Biointerphases, 2007).

However, a composition for excellently reducing the toxicity of nanoparticles and particulate matters has not been developed.

Therefore, there is a need for research on compositions capable of inhibiting the toxicity of nanoparticles and particulate matters generated in the environment.

### DISCLOSURE

### Technical Problem

An object of the present disclosure is to provide a nanotoxicity inhibitory composition capable of mitigating intracellular ATP reduction, cell viability reduction, cellular inflammatory morphological changes, and cellular activity induced by nanoparticles and environmentally-derived particulate matters.

### Technical Solution

To achieve the above object, example embodiments of the present invention provide a nanotoxicity inhibitory composition including one selected from the group consisting of a peptide-based compound and an organic acid or a mixture thereof as an active ingredient.

In addition, example embodiments of the present invention provide a cosmetic composition, pharmaceutical composition, or health food composition for preventing or treating cytotoxicity induced by nano- or particulate-matters including the nanotoxicity inhibitory composition as an active ingredient.

### Advantageous Effects

A nanotoxicity inhibitory composition according to example embodiments of the present invention may mitigate intracellular ATP reduction, cell viability reduction, cellular inflammatory morphological changes, and cellular activity induced by nanoparticles or environmentally-derived particulate matters.

In addition, it is possible to provide a cosmetic composition, pharmaceutical composition, or health food composition for preventing or treating cytotoxicity induced by nanoparticles or environmentally-derived particulate matters by including the composition as an active ingredient.

### BRIEF DESCRIPTIONS OF THE DRAWINGS

FIG. 1 shows results of analyzing the amount of ATP in microglia treated with 0.01 μg/μl or 0.1 μg/μl of MNPs@SiO₂(RITC) (average diameter of 50 nm) for 24 hours, and the black image on the bar graph is a diagram showing captured luminescence that was actually observed (*P<0.05, vs. control, #P<0.05, vs. group treated with 0.1 μg/μl of MNPs@SiO₂(RITC) only).

FIG. 2 is a diagram showing results of performing cellular morphological analysis with an optical microscope after treating particle-untreated microglia with glutathione, citric acid, and a mixture of glutathione and citric acid for 24 hours.

FIG. 3 is a diagram showing results of performing cellular morphological analysis with fluorescence and optical microscopes after treating microglia with glutathione, citric acid, and a mixture of glutathione and citric acid together with 0.1 μg/μl of MNPs@SiO₂(RITC) for 24 hours (Red color represents RITC fluorescence of MNPs@SiO₂(RITC)).

FIG. 4 is a diagram showing results of performing cellular morphological analysis with an optical microscope after treating microglia with glutathione, citric acid, and a mixture of glutathione and citric acid together with 0.1 μg/μl of silica nanoparticles (SiO₂, average diameter of 50 nm) for 24 hours.

FIG. 5 is a diagram showing results of performing cellular morphological analysis with an optical microscope after treating microglia with glutathione, citric acid, and a mixture of glutathione and citric acid together with 0.1 μg/μl of silver nanoparticles (Ag, average diameter of 20 nm) for 24 hours.

FIG. 6 is a diagram showing cellular morphological analysis with an optical microscope after treating microglia with glutathione, citric acid, and a mixture of glutathione and citric acid together with 0.1 μg/μl of gold nanoparticles (Au, average diameter of 10 nm) for 24 hours.

FIG. 7 is a diagram showing cellular morphological analysis with fluorescence and optical microscopes after treating microglia with glutathione, citric acid, and a mixture of glutathione and citric acid together with 0.1 μg/μl of quantum dot nanoparticles (CdSe, average diameter of 10 nm) for 24 hours (Green color represents self-fluorescence of quantum dot nanoparticles (CdSe, average diameter of 10 nm)).

FIG. 8 is a diagram showing cellular morphological analysis with an optical microscope after treating microglia with glutathione, citric acid, and a mixture of glutathione and citric acid together with 0.1 μg/μl of polystyrene microplastics (PS, average diameter of 2 μm) for 24 hours.

FIG. 9 is a diagram showing cellular morphological analysis with an optical microscope after treating microglia with glutathione, citric acid, and a mixture of glutathione and citric acid together with 0.1 μg/μl of polystyrene microplastics (PS, average diameter of 100 nm) for 24 hours.

FIG. 10 is a diagram showing cellular morphological analysis by an optical microscope after treating microglia with glutathione, citric acid, and a mixture of glutathione and citric acid together with 0.1 μg/μl of urban particulate matters (UPM, NIST 1648A) for 24 hours.

FIG. 11 is a diagram showing cellular morphological analysis with an optical microscope after treating microglia with glutathione, citric acid, and a mixture of glutathione and citric acid together with 0.1 μg/μl of silica nanoparticles (SiO₂, average diameter of 30 nm) for 24 hours.

FIG. 12 is a diagram showing cellular morphological analysis with an optical microscope after treating microglia with glutathione, citric acid, and a mixture of glutathione and citric acid together with 0.1 μg/μl of titanium oxide nanoparticles (TiO₂, average diameter of 40 nm) for 24 hours.

FIG. 13 is a diagram showing cellular morphological analysis with an optical microscope after treating microglia with glutathione, citric acid, and a mixture of glutathione and citric acid together with 0.1 μg/μl of silica carbon nanotubes (MWCNT, average diameter of 25 nm) for 24 hours.

FIG. 14 is a diagram showing cell viability after treating microglia with glutathione, citric acid, and a mixture of glutathione and citric acid together with 0.1 μg/μl of MNPs@SiO₂(RITC) (average diameter of 50 nm), silica nanoparticles (SiO₂, average diameter of 50 nm), silver nanoparticles (Ag, average diameter of 20 nm), gold nanoparticles (Au, average diameter of 10 nm), quantum dot nanoparticles (CdSe, average diameter of 10 nm), polystyrene microplastics (PS, average diameter of 2 μm and 100 nm), urban particulate matters (UPM, NIST 1648A), silica nanoparticles (SiO₂, average diameter of 30 nm), titanium oxide nanoparticles (TiO₂, average diameter of 40 nm), and carbon nanotubes (MWCNT, average diameter of 25 nm) for 24 hours (*P<0.05, vs. control group, #P<0.05, vs. group treated with 0.1 μg/μl of MNPs@SiO₂(RITC) only).

FIGS. 15A to 15G are diagrams showing results of measuring the distribution of MNPs@SiO₂(RITC) distributed in the brain hippocampus area of the mice and the degree of activation of microglia by intraperitoneally injecting MNPs@SiO₂(RITC) and a mixture of glutathione and citric acid into a mouse model (*P<0.05, vs. control group, #P<0.05, vs. group treated with MNPs@SiO₂(RITC) only).

FIGS. 16A to 16F are diagrams showing results of measuring the distribution of MNPs@SiO₂(RITC) distributed in the brain thalamus area of the mice and the degree of activation of microglia by intraperitoneally injecting MNPs@SiO₂(RITC) and a mixture of glutathione and citric acid into a mouse model (*P<0.05, vs. control group, #P<0.05, vs. group treated with MNPs@SiO₂(RITC) only).

FIGS. 17A to 17F are diagrams showing results of measuring the distribution of MNPs@SiO₂(RITC) distributed in the brain cortex area of the mice and the degree of activation of microglia by intraperitoneally injecting MNPs@SiO₂(RITC) and a mixture of glutathione and citric acid into a mouse model (*P<0.05, vs. control group, #P<0.05, vs. group treated with MNPs@SiO₂(RITC) only).

FIGS. 18A to 18F are diagrams showing results of measuring the distribution of MNPs@SiO₂(RITC) distributed in the brain striatum area of the mice and the degree of activation of microglia by intraperitoneally injecting MNPs@SiO₂(RITC) and a mixture of glutathione and citric acid into a mouse model (*P<0.05, vs. control group, #P<0.05, vs. group treated with MNPs@SiO₂(RITC) only).

FIGS. 19A to 19F are diagrams showing results of measuring the distribution of MNPs@SiO₂(RITC) distributed in the brain cerebellum area of the mice and the degree of activation of microglia by intraperitoneally injecting MNPs@SiO₂(RITC) and a mixture of glutathione and citric acid into a mouse model (*P<0.05, vs. control group, #P<0.05, vs. group treated with MNPs@SiO₂(RITC) only).

### MODES FOR CARRYING OUT INVENTION

Hereinafter, the present invention will be described in detail.

The present inventors prepared a nanotoxicity inhibitory composition including glutathione (GSH) and citric acid as an active ingredient and completed the present invention by finding that the nanotoxicity inhibitory composition is capable of mitigating intracellular ATP reduction, cell viability reduction, inflammatory morphological changes, and cell activity induced by nanoparticles and particulate matters.

An example embodiment of the present invention provides a nanotoxicity inhibitory composition including one selected from the group consisting of a peptide-based compound and an organic acid or a mixture thereof as an active ingredient.

In this case, the peptide-based compound may be glutathione (GSH), and the organic acid may be citric acid, the composition may include the peptide-based compound and the organic acid in a concentration ratio of (0.05 to 10):1, but is not limited thereto.

In addition, the nanotoxicity inhibitory composition may inhibit intracellular toxicity induced by nanoparticles or environmentally-derived particulate matters and may mitigate intracellular ATP reduction, cell viability reduction, cellular inflammatory morphological changes, and cellular activity.

According to an example embodiment of the present invention, there is an effect that the intracellular ATP reduction by nanoparticles may be mitigated by glutathione, citric acid, and a mixture of glutathione and citric acid.

In general, the term “nanoparticles” refers to particles having an average diameter in the range of 1-100 nm, and particulate matters (PMs) are classified as matters with an average diameter of 10 μm (PM10) and ultrafine matters with an average diameter of 2.5 μm (PM2.5), wherein the nanoparticles or environmentally-derived particulate matters may be selected from the group consisting of magnetic nanoparticles, inorganic nanoparticles, metal nanoparticles, quantum dot nanoparticles, carbon nanotubes, microplastics, and urban particulate matters.

Specifically, the group may consist of silica-coated magnetic nanoparticles [MNPs@SiO₂(RITC)] including chemically bound rhodamine B isocyanate, silica nanoparticles, silver nanoparticles, gold nanoparticles, CdSe quantum dot nanoparticles, polystyrene microplastics, urban particulate matters (UPM, NIST 1648A), titanium oxide nanoparticles, and carbon nanotubes but is not limited thereto, and may include any nanoparticle or particulate matter.

According to an example embodiment of the present invention, there is an effect that inflammatory morphological changes of microglia may be mitigated by glutathione, citric acid, and a mixture of glutathione and citric acid, wherein the morphological change is caused by the MNPs@SiO₂(RITC) (average diameter of 50 nm), silica nanoparticles (SiO₂, average diameter of 50 nm), silver nanoparticles (Ag, average diameter of 20 nm), gold nanoparticles (Au, average diameter of 10 nm), quantum dot nanoparticles (CdSe, average diameter of 10 nm), polystyrene microplastics (PS, average diameter of 2 μm and 100 nm), urban particulate matters (UPM, NIST 1648A), silica nanoparticles (SiO₂, average diameter of 30 nm), titanium oxide nanoparticles (TiO₂, average diameter of 40 nm), and carbon nanotubes (MWCNT, average diameter of 25 nm).

In addition, there is an effect that the cell viability reduction of microglia may be mitigated by glutathione, citric acid, and a mixture of glutathione and citric acid, wherein the cell viability reduction is caused by the MNPs@SiO₂(RITC) (average diameter of 50 nm), silica nanoparticles (SiO₂, average diameter of 50 nm), silver nanoparticles (Ag, average diameter of 20 nm), gold nanoparticles (Au, average diameter of 10 nm), quantum dot nanoparticles (CdSe, average diameter of 10 nm), polystyrene microplastics (PS, average diameter of 2 μm and 100 nm), urban particulate matters (UPM, NIST 1648A), silica nanoparticles (SiO₂, average diameter of 30 nm), titanium oxide nanoparticles (TiO₂, average diameter of 40 nm), and carbon nanotubes (MWCNT, average diameter of 25 nm).

Further, there is an effect that the decrease in the filament length of microglia and the increase in protein (Iba1, CD40, CD11b) expression level which increases in accordance with activation of cells may be mitigated by glutathione, citric acid, and a mixture of glutathione and citric acid, wherein the decrease and the increase are caused by the MNPs@SiO₂(RITC) (average diameter of 50 nm), silica nanoparticles (SiO₂, average diameter of 50 nm), silver nanoparticles (Ag, average diameter of 20 nm), gold nanoparticles (Au, average diameter of 10 nm), quantum dot nanoparticles (CdSe, average diameter of 10 nm), polystyrene microplastics (PS, average diameter of 2 μm and 100 nm), urban particulate matters (UPM, NIST 1648A), silica nanoparticles (SiO₂, average diameter of 30 nm), titanium oxide nanoparticles (TiO₂, average diameter of 40 nm), and carbon nanotubes (MWCNT, average diameter of 25 nm).

In addition, the cell is selected from the group consisting of microglia, neurons, astrocytes, and oligodendrocytes, but is not limited thereto.

In addition, an example embodiment of the present invention provides a cosmetic composition for preventing or treating cytotoxicity induced by nano-or particulate-matters including the nanotoxicity inhibitory composition as an active ingredient.

If the composition of an example embodiment of the present invention is a cosmetic composition, the cosmetic composition may include a conventional adjuvant such as stabilizers, solubilizers, vitamin, pigments, and fragrances and a carrier in addition to glutathione (GSH) or citric acid or a mixture thereof which are active ingredients.

The formulation of the cosmetic composition may be prepared in any formulation conventionally prepared in the art, for example, hair tonic, hair conditioner, hair essence, hair lotion, hair nourishment lotion, hair shampoo, hair conditioner, hair treatment, hair cream, hair nourishment cream, hair moisturizing cream, hair massage cream, hair wax, hair aerosol, hair pack, hair nourishment pack, hair soap, hair cleansing foam, hair oil, hair drying agent, hair preservative, hair dye, hair waving agent, hair bleaching agent, hair gel, hair glaze, hair dressing agent, hair lacquer, hair moisturizer, hair mousse, and hair spray, but is not limited thereto.

If the formulation is a paste, cream or gel, animal oil, vegetable oil, wax, paraffin, starch, tracanth, cellulose derivatives, polyethylene glycol, silicone, bentonite, silica, talc, or zinc oxide may be used as a carrier component.

If the formulation is powder or a spray, lactose, talc, silica, aluminum hydroxide, calcium silicate or polyamide powder may be used as a carrier component, and in particular, in the case of the spray, chlorofluorohydrocarbon, propane/butane or a booster such as dimethyl ether may be additionally included.

If the formulation is a solution or emulsion, solvents, solubilizers or emulsifiers are used as a carrier component, for example, water, ethanol, isopropanol, ethyl carbonate, ethyl acetate, benzyl alcohol, benzyl benzoate, propylene glycol, 1,3-butylglycol oil, glycerol fatty ester, polyethylene glycol or fatty acid ester of sorbitan.

If the formulation is a suspension, a liquid diluent such as water, ethanol or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tracanth may be used as a carrier component.

In addition, an example embodiment of the present invention provides a pharmaceutical composition for preventing or treating cytotoxicity induced by nano-or particulate-matters including the nanotoxicity inhibitory composition as an active ingredient.

If the composition of an example embodiment of the present invention is a pharmaceutical composition, the pharmaceutical composition may be formulated as a cream, gel, patch, spray, ointment, emplastrum, lotion, liniment agent, pasta agent, and cataplasmas. In addition, the pharmaceutical composition may include a pharmaceutically acceptable carrier in addition to the active ingredient, and such pharmaceutically acceptable carriers are conventionally used in pharmaceutical formulations, wherein the pharmaceutically acceptable carriers may include lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, but are not limited thereto. Further, the pharmaceutical composition may further include lubricants, wetting agents, sweetening agents, flavoring agents, emulsifying agents, suspending agents, and preservatives as an additive.

An administration method of the pharmaceutical composition is determined by the severity of symptoms while a topical administration method is generally preferred. In addition, the dosage of the active ingredient in the pharmaceutical composition may vary depending on the route of administration, the degree of disease, and the age, sex, and weight of a patient, and may be administered once to several times a day.

In addition, an example embodiment of the present invention provides a health food composition for preventing or treating cytotoxicity induced by nano-or particulate-matters including the nanotoxicity inhibitory composition as an active ingredient.

The health food composition may be provided in the form of powder, granules, tablets, capsules, syrups or beverages, and the health food composition is used together with other foods or food additives in addition to glutathione (GSH) or citric acid or a mixture thereof according to an example embodiment of the present invention which are active ingredients and may be appropriately used according to a conventional method. The mixed amount of the active ingredient may be suitably determined depending on the purpose of use thereof, for example, prevention, health or therapeutic treatment.

While the effective dose of glutathione (GSH) or citric acid or a mixture thereof included in the health food composition may be used according to the effective dose of the pharmaceutical composition, for health and hygiene purposes or long-term intake pursuing health control, the dose may be less than the above range, and it is certain that the active ingredient can be used in an amount above the range since there is no problem in terms of safety.

The type of health food is not particularly limited, and examples may include meat, sausage, bread, chocolate, candy, snacks, confectionery, pizza, ramen, other noodles, gum, dairy products including ice cream, various soups, beverages, tea, drinks, alcoholic beverages, and vitamin complexes.

While the major toxicity of nanoparticles and particulate matters is derived by the reduction in energy metabolism due to an increase of active oxygen and mitochondrial damage, a composition has been developed in the present disclosure, which efficiently inhibits toxicity owing to an antioxidant effect of glutathione as well as a synergistic effect of energy metabolism promotion by citric acid and metal ion chelation.

According to an example embodiment of the present invention, ATP reduction, apoptosis, cellular morphological changes and cell activity were observed in rat primary microglia treated with the nanoparticles and particulate matters, and it was confirmed that intracellular ATP was increased, cell viability was increased, and cell activation was reduced due to glutathione, citric acid, and the mixture of glutathione and citric acid, thereby being able to be used as a material for reducing toxicity of the nanoparticles and particulate matters.

Hereinafter, examples will be described in detail to help the understanding of the present invention. However, the following examples are merely illustrative of the content of the present invention, and the scope of the present invention is not limited to the following examples. The examples of the present invention are provided to more completely explain the present invention to those skilled in the art.

### <Reference Example> Experimental Material

Silica-coated magnetic nanoparticles including chemically bound rhodamine B isothiocyanate [MNPs@SiO₂(RITC), average diameter of 50 nm] were obtained from BITERIALS (Korea), silica nanoparticles (SiO₂, average diameter of 50 nm) from Seo H, Kim S-W (2007) In Situ Synthesis of CdTe/CdSe Core-Shell Quantum Dots. Chemistry of Materials 19:2715-2717; Kim J, Lee JE, Lee J, Jang Y, Kim S-W, An K, Yu JH, Hyeon T (2006a) Generalized Fabrication of Multifunctional Nanoparticle Assemblies on Silica Spheres. Angew Chem 45:4789-4793, silver nanoparticles (Ag, average diameter of 20 nm) from Kim et al, 2006a; Seo & Kim, 2007, gold nanoparticles (Au, average diameter of 10 nm) from Kim et al, 2006a; Seo & Kim, 2007, quantum dot nanoparticles (CdSe, average diameter of 10 nm) from Kim et al, 2006a; Seo & Kim, 2007, polystyrene microplastics (PS, mean diameter of 2 μm and 100 nm) from Sigma-Aldrich (USA), urban particulate matters (UPM, NIST 1648A) from Sigma-Aldrich (USA), silica nanoparticles (SiO₂, average diameter of 30 nm) from US Research Nanomaterials (USA), titanium oxide nanoparticles (TiO₂, average diameter of 40 nm) from US Research Nanomaterials (USA), and carbon nanotubes (MWCNT, average diameter of 25 nm) from US Research Nanomaterials (USA).

### <Example 1> Measurement of Adenosine Triphosphate (ATP) in Microglia Treated with MNPs@SiO₂(RITC)

1. Cell culture

Brain tissues of 1-day-old rat were removed, and only microglia were isolated from the removed brain tissues.

The cells were suspended in Minimum Essential Medium Eagle (MEM) containing 10% fetal bovine serum, 100 units/ml of penicillin, and 100 ng/μl of streptomycin. Then, the cells were cultured in an incubator at 37° C. in the presence of 5% CO₂.

2. Measurement of intracellular ATP

Microglia in culture were treated with 0.01 μg/μl or 0.1 μg/μl of MNPs@SiO₂(RITC) (average diameter of 50 nm) for 24 hours. At this time, in the case of groups of glutathione, citric acid, and a mixture of glutathione and citric acid, nanoparticles were treated together. After the cells were suspended, the number of cells was measured and adjusted to the same cell number. The cells became luminescent according to the amount of ATP using a luciferin-based ATP luminescence measurement kit (Promega, USA), the luminescence degree was measured by a luminometer (LMaxII384; Molecular Devices, USA), and ChemiDoc™ Touch Gel Imaging System (Bio-Rad) was used for imaging.

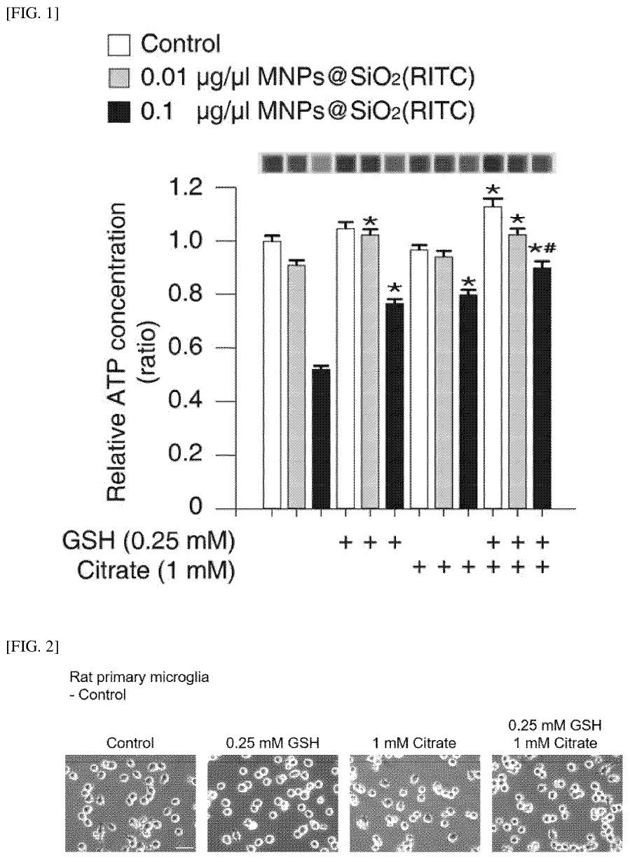

As a result, when glutathione or citric acid was added together with the mixture of glutathione and citric acid, it was confirmed that ATP reduction was inhibited compared to a group that microglia which is in culture was treated only with 0.01 μg/μl or 0.1 μg/μl of MNPs@SiO₂(RITC) (average diameter of 50 nm), and 30%, 30%, and 45% increases respectively were checked compared to a group treated only with 0.1 μg/μl of MNPs@SiO₂(RITC) (average diameter of 50 nm) (FIG. 1).

A black image on a bar graph in FIG. 1 was generated by capturing actually observed luminescence, meaning that the closer to black, the higher the ATP amount, satisfactorily matching with the result of the bar graph, and confirming that the ATP reduction was inhibited upon addition of glutathione or citric acid or a mixture of glutathione and citric acid.

### <Example 2> Morphological Analysis of Microglia Treated with Nanoparticles and Particulate Matters

### 1. Morphological Analysis

Microglia cultured by the cell culture method of Example 1 was treated with 0.1 μg/μl of MNPs@SiO₂(RITC) (average diameter of 50 nm), silica nanoparticles (SiO₂, average diameter of 50 nm), silver nanoparticles (Ag, average diameter of 20 nm), gold nanoparticles (Au, average diameter of 10 nm), quantum dot nanoparticles (CdSe, average diameter of 10 nm), polystyrene microplastics (PS, average diameter of 2 μm and 100 nm), urban particulate matters (UPM, NIST 1648A), silica nanoparticles (SiO₂, average diameter of 30 nm), titanium oxide nanoparticles (TiO₂, average diameter of 40 nm), and carbon nanotubes (MWCNT, average diameter of 25 nm) for 24 hours, and then images were taken using fluorescence and optical microscopes (Axio Vert 200M fluorescence microscopy, Zeiss, Jena, Germany). Fluorescence of MNPs@SiO₂(RITC) (average diameter of 50 nm) and quantum dot nanoparticles (CdSe, average diameter of 10 nm) which are fluorescent were photographed also. Changes in the number of microglia treated with nanoparticles and particulate matters, normal morphology (cell branched state), inflammatory morphology (round-shaped state), and abnormal morphology (form embedded in a particle) were observed.

As a result, when particle-untreated microglia were treated with glutathione, citric acid, and the mixture of glutathione and citric acid for 24 hours, and then cellular morphology was analyzed with an optical microscope, it was confirmed that there was no change in cell number or morphology (FIG. 2).

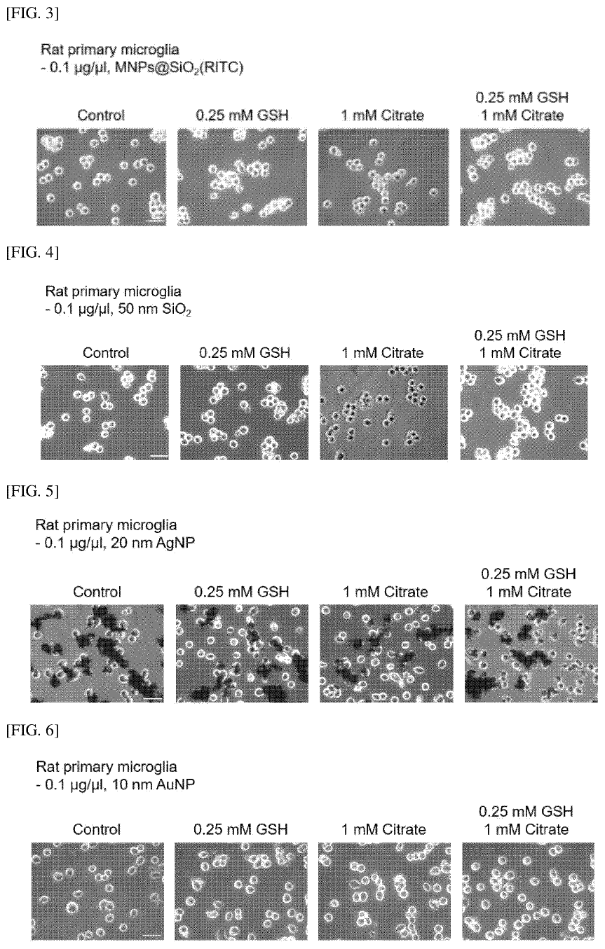

In addition, when microglia were treated with glutathione, citric acid, and the mixture of glutathione and citric acid together with 0.1 μg/μl of MNPs@SiO₂(RITC) for 24 hours and then cellular morphological analysis was performed with fluorescence and optical microscopes, it was confirmed that the decrease in the cell number due to MNPs@SiO₂(RITC) was mitigated by glutathione or citric acid while the greatest mitigating effect was shown in the mixture of glutathione and citric acid (FIG. 3).

In addition, when microglia were treated with glutathione, citric acid, and the mixture of glutathione and citric acid together with 0.1 μg/μl silica nanoparticles (SiO₂, average diameter of 50 nm) for 24 hours, and then cellular morphological analysis was performed with an optical microscope, it was confirmed that the decrease in the cell number due to silica nanoparticles (SiO₂, average diameter of 50 nm) was mitigated by glutathione or citric acid, while the most mitigating effect was shown in the mixture of glutathione and citric acid (FIG. 4).

In addition, when microglia were treated with glutathione, citric acid, and the mixture of glutathione and citric acid together with 0.1 μg/μl of silver nanoparticles (Ag, average diameter of 20 nm) for 24 hours, and then cellular morphological analysis was performed with an optical microscope, the decrease in the cell number due to silver nanoparticles (Ag, average diameter of 20 nm) was mitigated by glutathione or citric acid, while the most mitigating effect was shown in the mixture of glutathione and citric acid. Morphologically, it was confirmed that the group treated with the mixture of glutathione and citric acid entered an inactive (normal) state (FIG. 5).

In addition, when microglia were treated with glutathione, citric acid, and the mixture of glutathione and citric acid together with 0.1 μg/μl of gold nanoparticles (Au, average diameter of 10 nm) for 24 hours, and then cellular morphological analysis was performed with an optical microscope, the decrease in the cell number due to gold nanoparticles (Au, average diameter of 10 nm) was mitigated by glutathione or citric acid, while the most mitigating effect was shown in the mixture of glutathione and citric acid. Morphologically, it was confirmed that the group treated with the mixture of glutathione and citric acid entered an inactive (normal) state (FIG. 6).

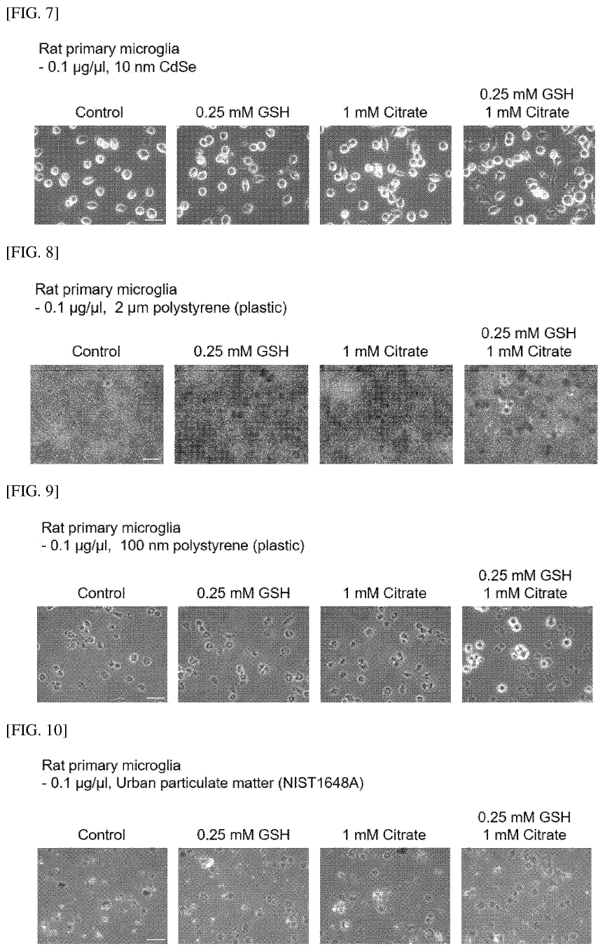

In addition, when microglia were treated with glutathione, citric acid, and the mixture of glutathione and citric acid together with 0.1 μg/μl of quantum dot nanoparticles (CdSe, average diameter of 10 nm) for 24 hours, and then cellular morphological analysis was performed with fluorescence and optical microscopes, the decrease in the cell number due to quantum dot nanoparticles (CdSe, average diameter of 10 nm) was mitigated by glutathione or citric acid, while the most mitigating effect was shown in the mixture of glutathione and citric acid. Morphologically, it was confirmed that the group treated with the mixture of glutathione and citric acid entered an inactive (normal) state (FIG. 7).

In addition, when microglia were treated with glutathione, citric acid, and the mixture of glutathione and citric acid together with 0.1 μg/μl of polystyrene microplastics (PS, average diameter of 2 μm) for 24 hours, and then cellular morphological analysis was performed with an optical microscope, the decrease in the cell number due to microplastics (PS, average diameter of 2 μm) was mitigated by glutathione or citric acid, while the most mitigating effect was shown in the mixture of glutathione and citric acid. Morphologically, an inactive (normal) state was observed in the group treated with the mixture of glutathione and citric acid (FIG. 8).

In addition, when microglia were treated with glutathione, citric acid, and the mixture of glutathione and citric acid together with 0.1 μg/μl of polystyrene microplastics (PS, average diameter of 100 nm) for 24 hours, and then cellular morphological analysis was performed with an optical microscope, the decrease in the cell number due to microplastics (PS, average diameter of 100 nm) was mitigated by glutathione or citric acid, while the most mitigating effect was shown in the mixture of glutathione and citric acid (FIG. 9) .

In addition, when microglia were treated with glutathione, citric acid, and the mixture of glutathione and citric acid together with 0.1 μg/μl of urban particulate matters (UPM, NIST 1648A) for 24 hours, and then cellular morphological analysis was performed with an optical microscope, the decrease in the cell number due to urban particulate matters (UPM, NIST 1648A) was mitigated by glutathione or citric acid, while the most mitigating effect was shown in the mixture of glutathione and citric acid (FIG. 10).

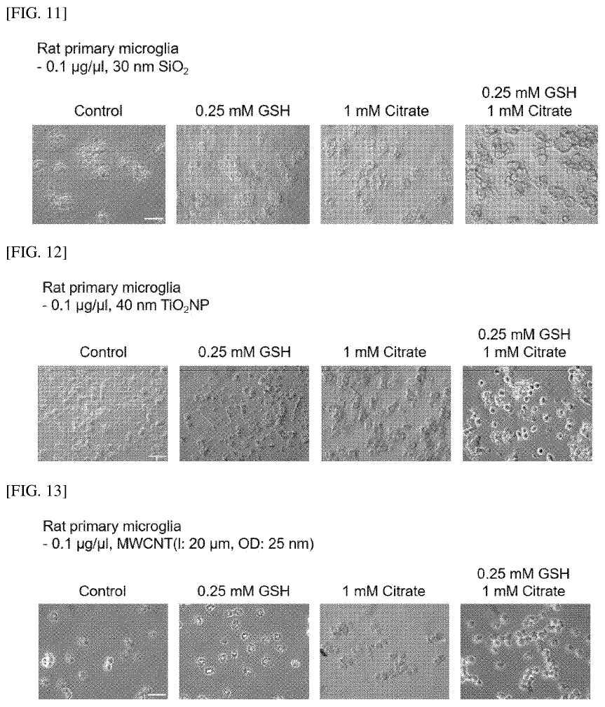

When microglia were treated with glutathione, citric acid, and the mixture of glutathione and citric acid together with 0.1 μg/μl of silica nanoparticles (SiO₂, average diameter of 30 nm) for 24 hours, and then cellular morphological analysis was performed with an optical microscope, the decrease in the cell number due to silica nanoparticles (SiO₂, average diameter of 30 nm) was mitigated by glutathione or citric acid, while the most mitigating effect was shown in the mixture of glutathione and citric acid. Morphologically, an inactive (normal) state was observed in the group treated with the mixture of glutathione and citric acid (FIG. 11).

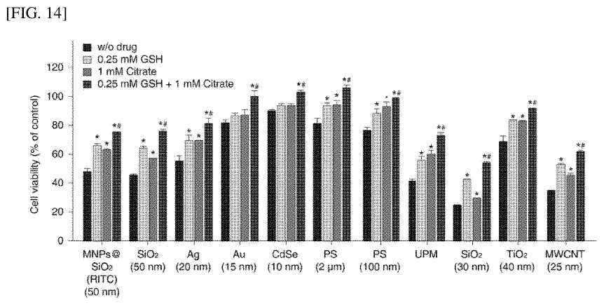

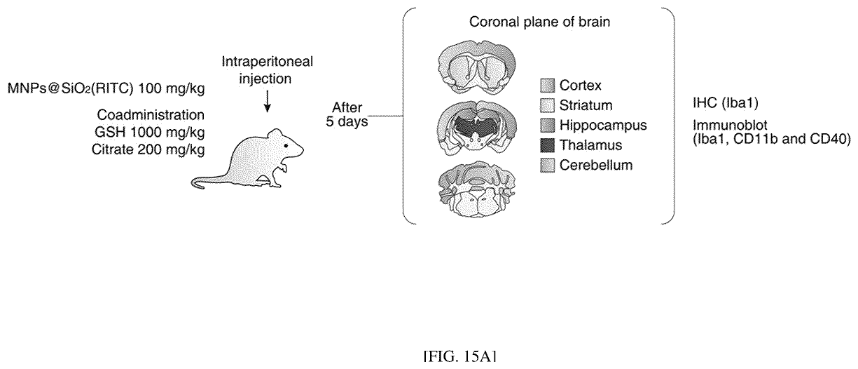

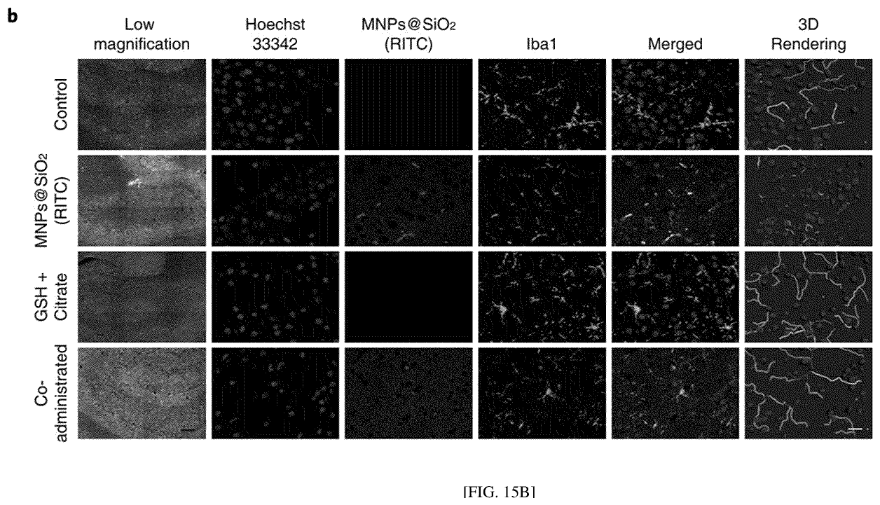

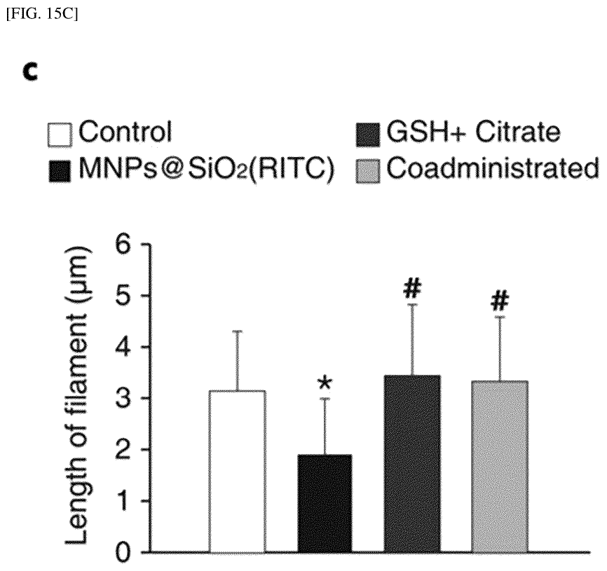

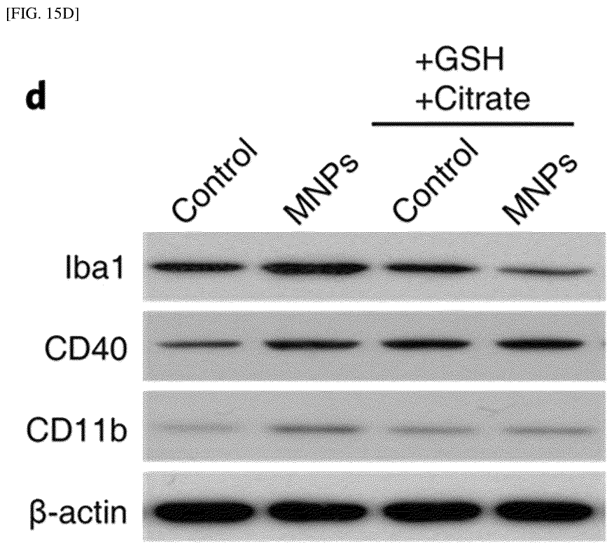

When microglia were treated with glutathione, citric acid, and the mixture of glutathione and citric acid together with 0.1 μg/μl of titanium oxide nanoparticles (TiO₂, average diameter of 40 nm) for 24 hours, and then cellular morphological analysis was performed with an optical microscope, the decrease in the cell number due to titanium oxide nanoparticles (TiO₂, average diameter of 40 nm) was mitigated by glutathione or citric acid, while the most mitigating effect was shown in the mixture of glutathione and citric acid. Morphologically, a distinct inactive (normal) state was observed in the group treated with the mixture of glutathione and citric acid (FIG. 12).

When microglia were treated with glutathione, citric acid, and the mixture of glutathione and citric acid together with 0.1 μg/μl of silica carbon nanotubes (MWCNT, average diameter of 25 nm) for 24 hours, and then cellular morphological analysis was performed with an optical microscope, the decrease in the cell number due to carbon nanotubes (MWCNT, average diameter of 25 nm) was mitigated by glutathione or citric acid, while the most mitigated effect was shown in the mixture of glutathione and citric acid. Morphologically, an inactive (normal) state was observed in the group treated with the mixture of glutathione and citric acid (FIG. 13).

### <Example 3> Measurement of Cell Viability of Microglia Treated with Nanoparticles and Particulate Matters

1. Cell viability analysis

Microglia cultured by the cell culture method of Example 1 were treated with 0.1 μg/μl of MNPs@SiO₂(RITC) (average diameter of 50 nm), silica nanoparticles (SiO₂, average diameter of 50 nm), silver nanoparticles (Ag, average diameter of 20 nm), gold nanoparticles (Au, average diameter of 10 nm), quantum dot nanoparticles (CdSe, average diameter of 10 nm), polystyrene microplastics (PS, average diameter of 2 μm and 100 nm), urban particulate matters (UPM, NIST 1648A), silica nanoparticles (SiO₂, average diameter of 30 nm), titanium oxide nanoparticles (TiO₂, average diameter of 40 nm), and carbon nanotubes (MWCNT, average diameter of 25 nm) for 24 hours, a kit (CellTilter 96 Aqueous One Solution Cell Proliferation Assay, Promega Corporation, Madison, WI) for analyzing cell viability based on the activity of succinic dehydrogenase was mixed, and the degree of formazan formation in accordance with the cell viability was measured with absorbance at 490 nm.

As a result, it was confirmed that the reduction in cell viability due to nanoparticles and particulate matters was mitigated by glutathione or citric acid, and the most mitigating effect was shown in the mixture of glutathione and citric acid (FIG. 14).

### <Example 4> Measurement of Microglial Activation in the Mouse Brain Treated with Nanoparticles

MNPs@SiO₂(RITC) and the mixture of glutathione and citric acid were intraperitoneally injected into a mouse model to measure the distribution of MNPs@SiO₂(RITC) and the degree of microglial activation in the mouse brain.

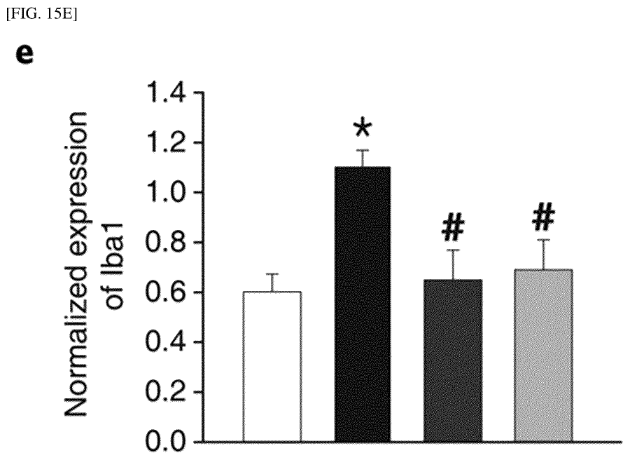

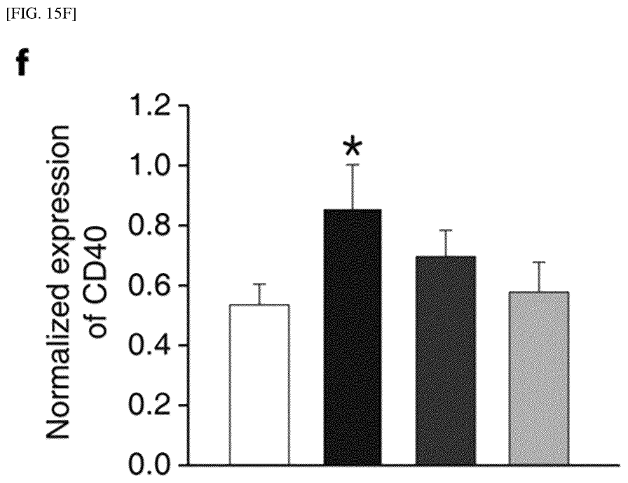

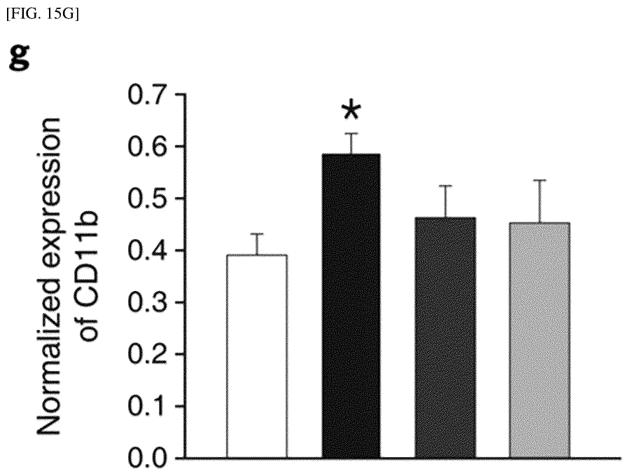

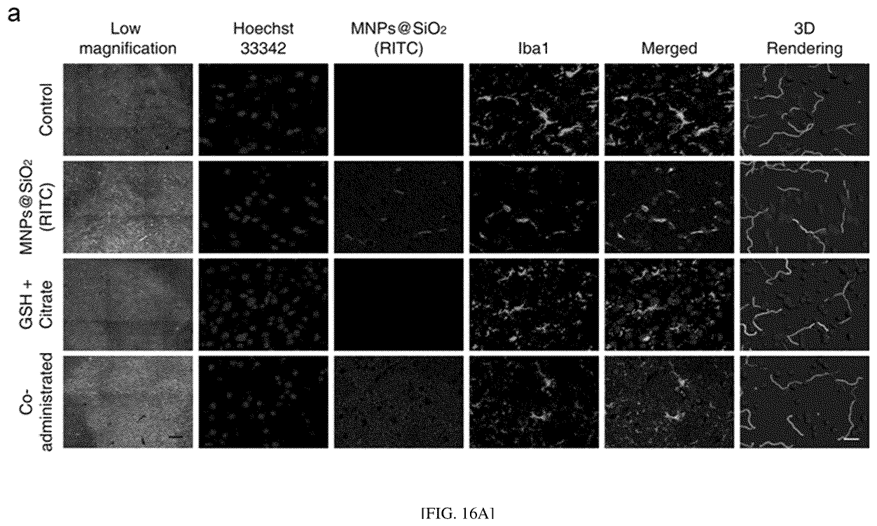

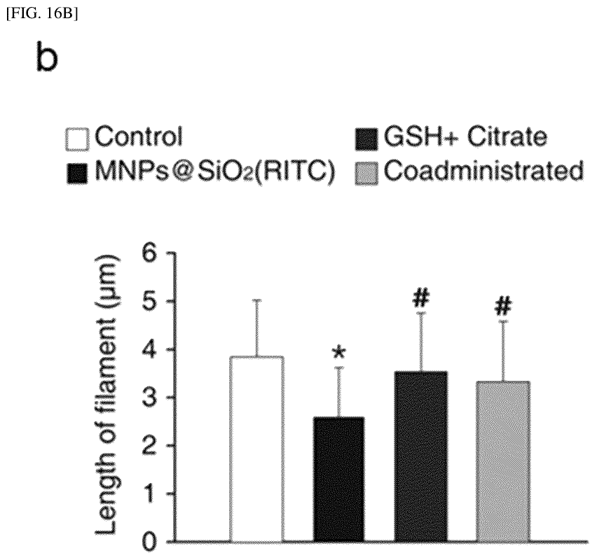

After intraperitoneal injection of 100 mg/kg of MNPs@SiO₂(RITC) and the mixture of glutathione (1000 mg/kg) and citric acid (200 mg/kg) into 8-week-old ICR mice, the mice were perfused with paraformaldehyde 5 days later, and the brain was removed and separated into cortex, striatum, hippocampus, thalamus, and cerebellum so as to be analyzed by immunohistochemistry (IHC) and immunoblot (FIG. 15A).

### 1. Immunohistochemical Analysis

The removed brain tissue was frozen sectioned and blocked with 1% bovine serum albumin and 10% donkey serum at room temperature for 2 hours. Anti-Iba1 polyclonal goat antibody (1:100) was bound to the blocked tissue at 4° C. for 16 hours. After washing the tissue with phosphate buffered saline containing 0.4% Triton X-100, Alexa Fluor 488-bound anti-goat IgG antibody (1:100) was subjected to the binding at room temperature for 2 hours. The tissue was washed with phosphate buffered saline containing 0.4% Triton X-100 and then sealed with a cover glass using a DAPI-included encapsulant. The stained tissue was observed and Z-stack scanned using a slide scanner (Axio Scan Z1, Zeiss, Germany) or a confocal microscope (Nikon A1R HD25, Japan). The scanned images were constructed into a 3D rendering model via the Imaris 9.2 (Bitplane, Zurich, Switzerland) program. The length of filaments of microglia was quantified in the constructed model.

FIG. 15B is a result of immunohistochemical analysis for the morphology of microglia and MNPs@SiO₂(RITC) distributed in the brain hippocampus area of the mice co-administrated with MNPs@SiO₂(RITC) and the mixture of glutathione and citric acid. Microglia were detected with a protein marker Iba1. The morphology of Iba1-stained microglia was constructed into a 3D rendering model via the Imaris 9.2 (Bitplane, Zurich, Switzerland) program, and the decrease in filament length (microglia activation) thereby was quantitatively analyzed (FIG. 15C).

Compared to the control group, the filament length of microglia distributed in the brain hippocampus area of the mice treated only with MNPs@SiO₂(RITC) was statistically, significantly decreased, and it was confirmed that such decrease was mitigated in mice co-administrated with the mixture of glutathione and citric acid.

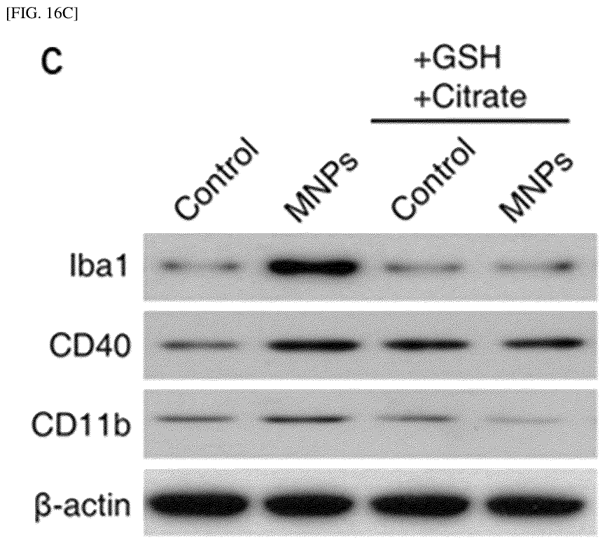

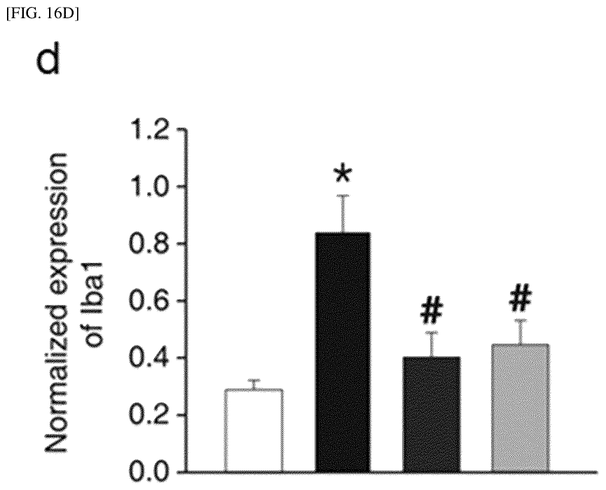

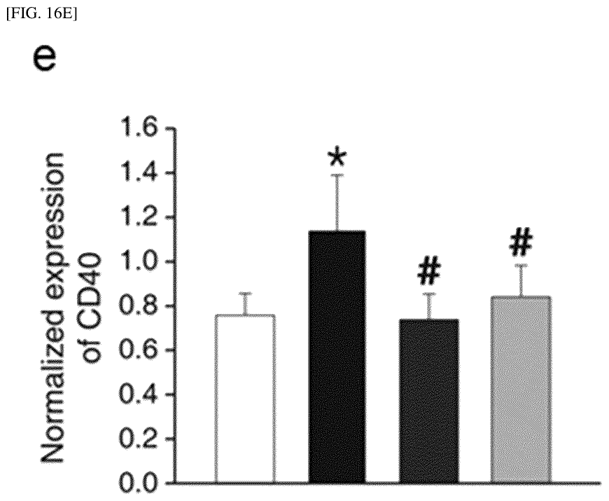

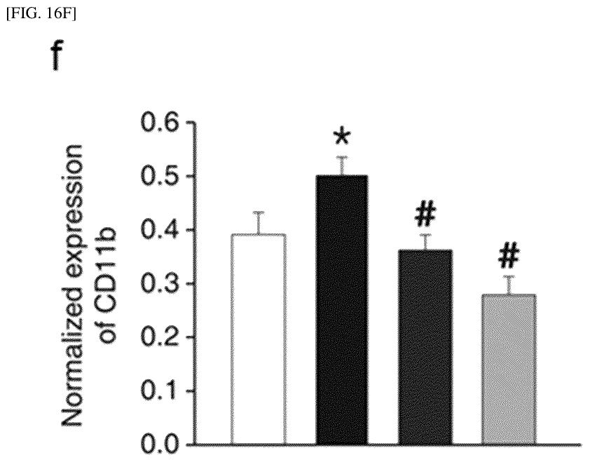

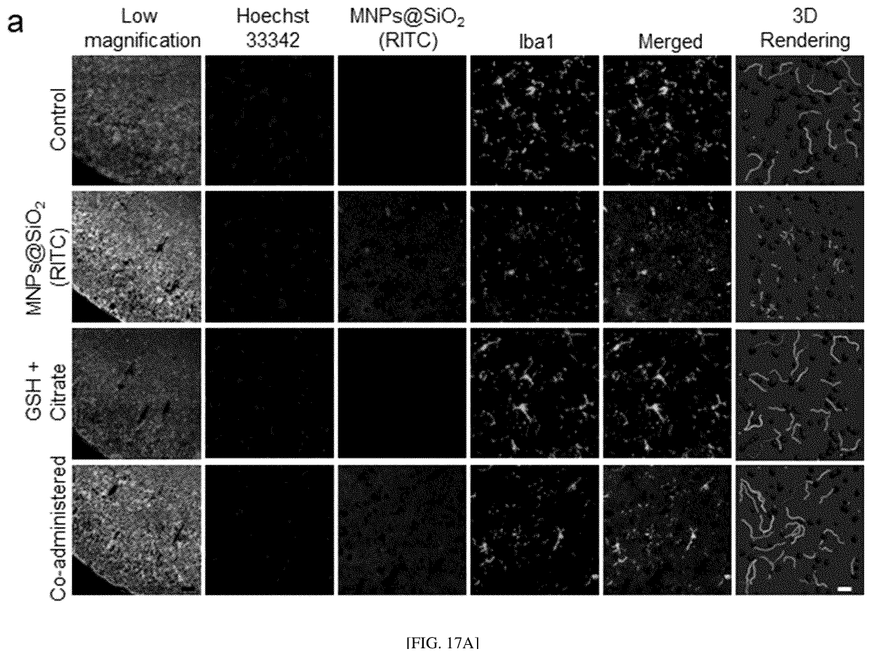

FIG. 16A is a result of immunohistochemical analysis of the morphology of MNPs@SiO₂(RITC) and microglia distributed in the brain thalamus area of the mice co-administrated with MNPs@SiO₂(RITC) and the mixture of glutathione and citric acid. The decrease in filament length was quantitatively analyzed by treating MNPs@SiO₂(RITC) (FIG. 16B).

Compared to the control group, the filament length of microglia distributed in the brain thalamus area of the mice treated only with MNPs@SiO₂ (RITC) was statistically, significantly decreased, and it was confirmed that such decrease was mitigated in mice co-administrated with the mixture of glutathione and citric acid.

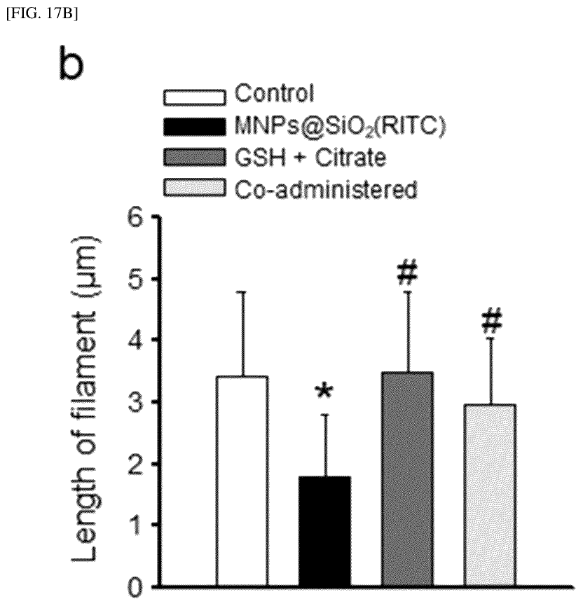

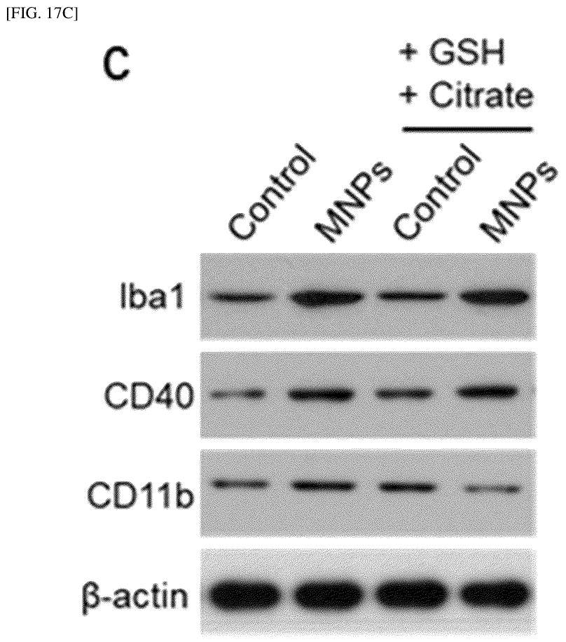

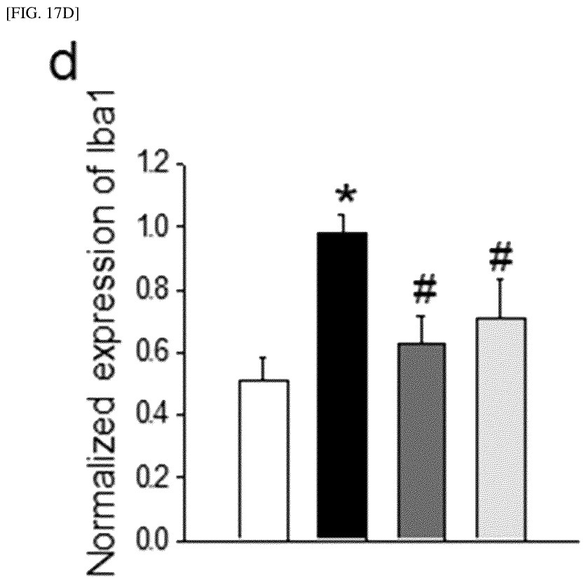

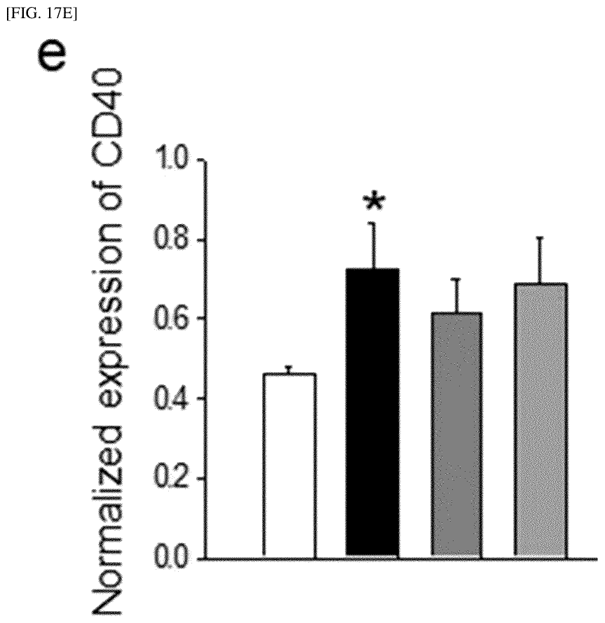

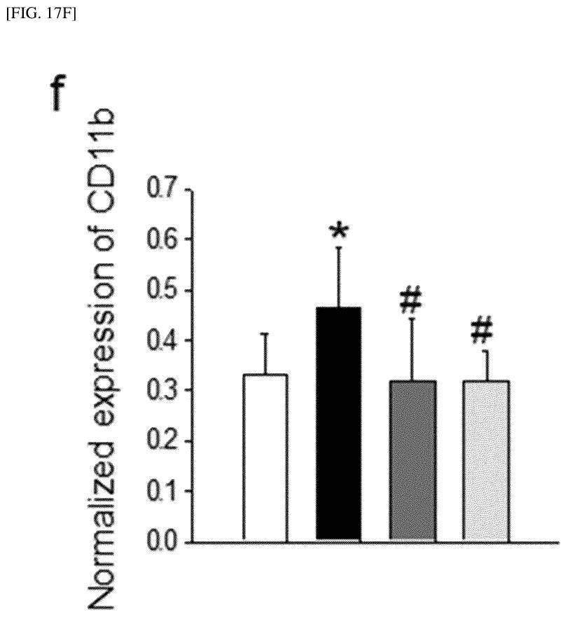

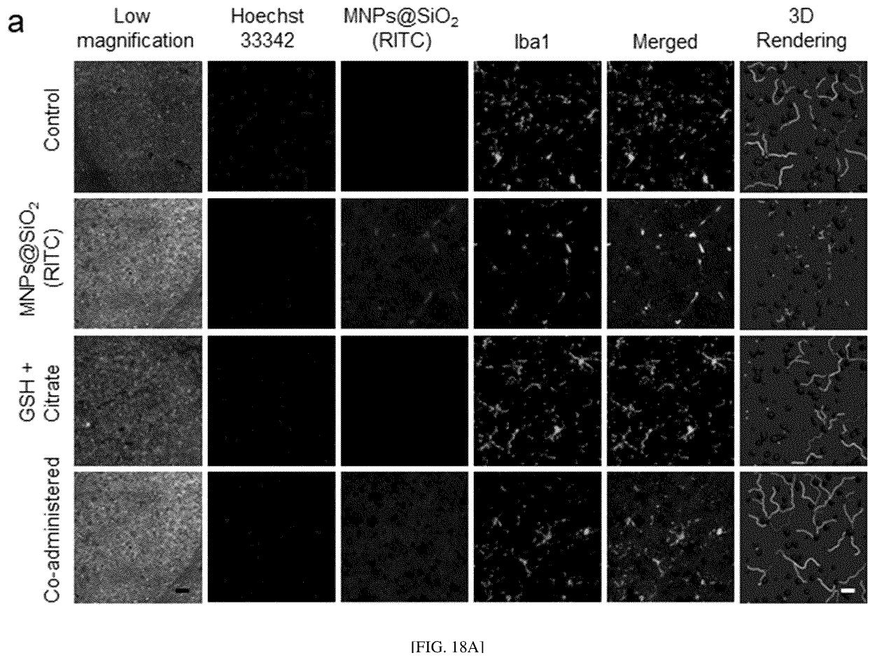

FIG. 17A is a result of an immunohistochemical analysis of the morphology of MNPs@SiO₂(RITC) and microglia distributed in the brain cortex area of the mice co-administrated with MNPs@SiO₂(RITC) and the mixture of glutathione and citric acid. The decrease in filament length was quantitatively analyzed by treating MNPs@SiO₂(RITC) (FIG. 17B).

Compared to the control group, the filament length of microglia distributed in the brain cortex area of the mice treated only with MNPs@SiO₂ (RITC) was statistically, significantly decreased, and it was confirmed that such decrease was mitigated in mice co-administrated with the mixture of glutathione and citric acid.

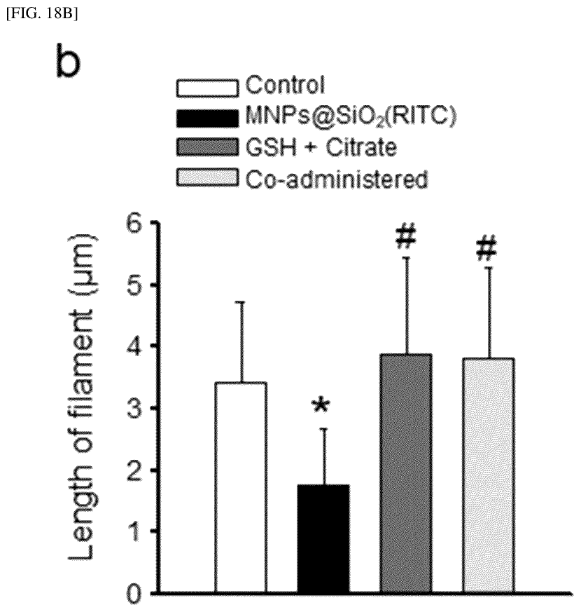

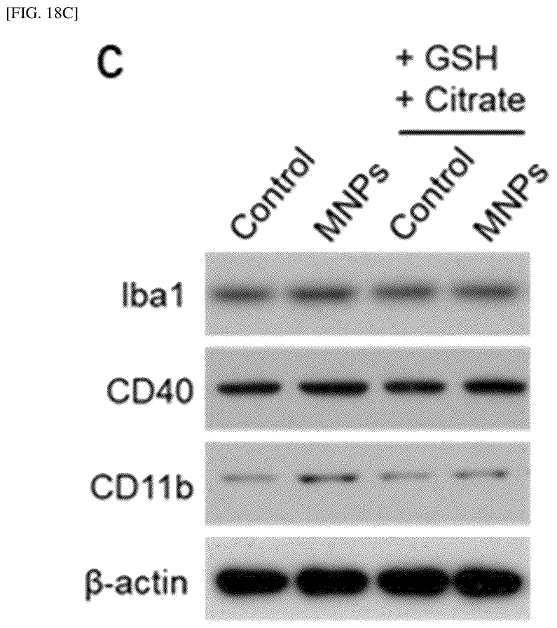

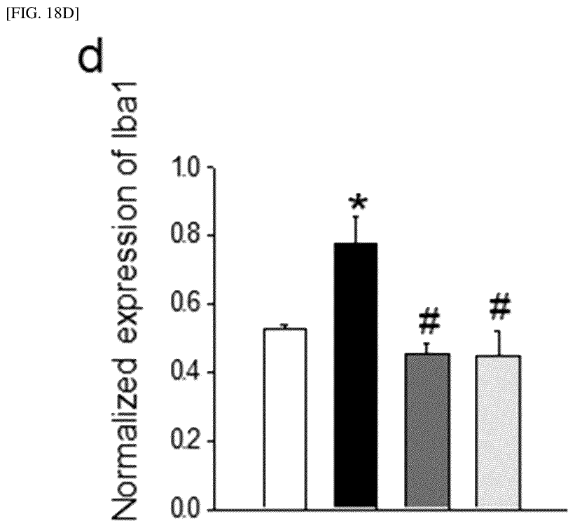

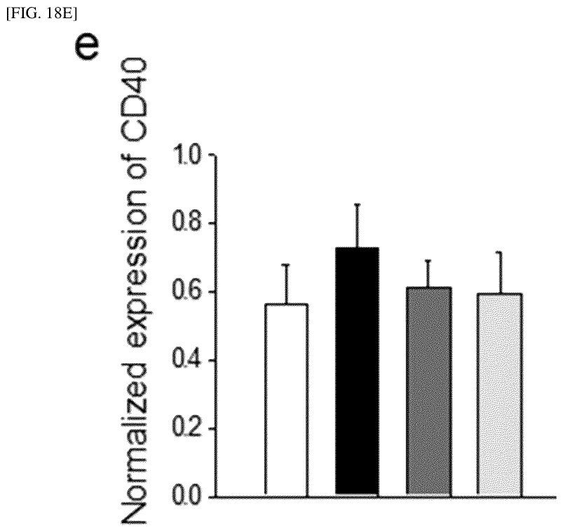

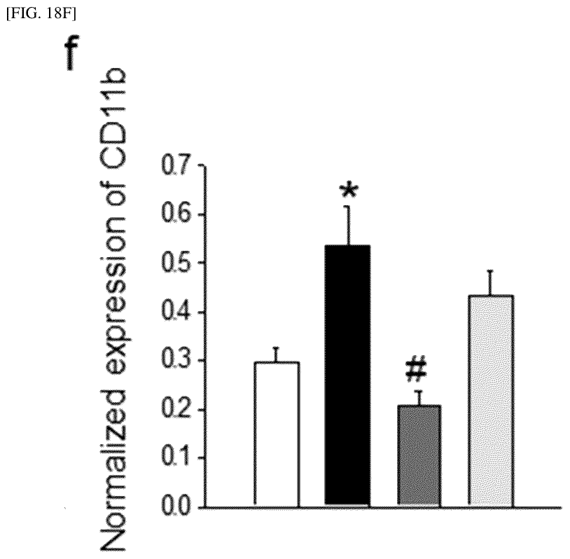

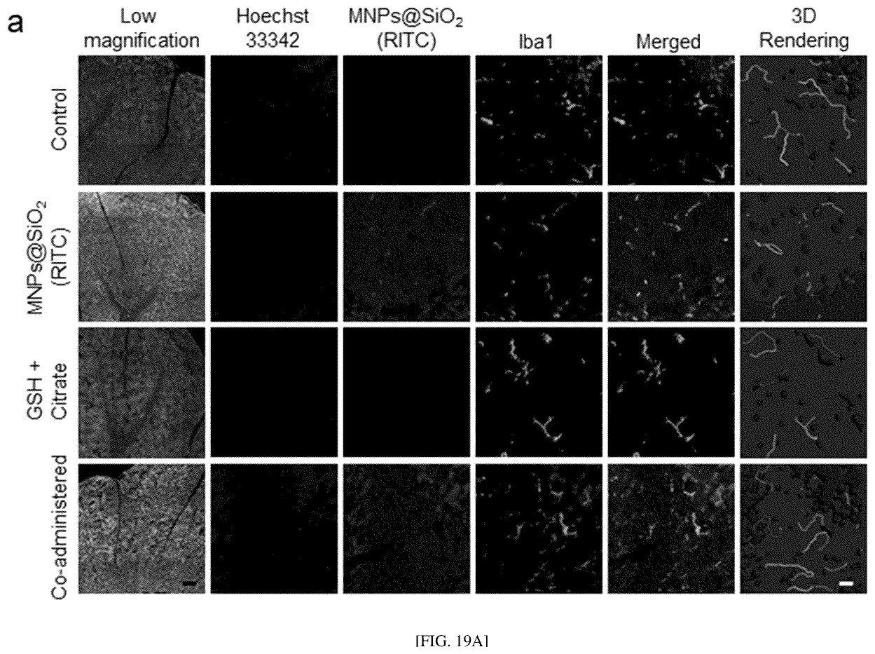

FIG. 18A is a result of immunohistochemical analysis of the morphology of MNPs@SiO₂(RITC) and microglia distributed in the brain striatum area of the mice co-administrated with MNPs@SiO₂(RITC) and the mixture of glutathione and citric acid. The decrease in filament length was quantitatively analyzed by treating MNPs@SiO₂(RITC) (FIG. 18B).

Compared to the control group, the filament length of microglia distributed in the brain striatum area of the mice treated only with MNPs@SiO₂(RITC) was statistically, significantly decreased, and it was confirmed that such decrease was mitigated in mice co-administrated with the mixture of glutathione and citric acid.

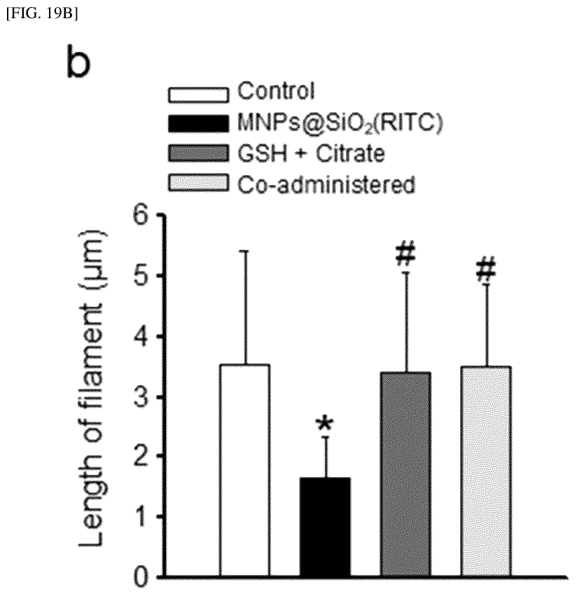

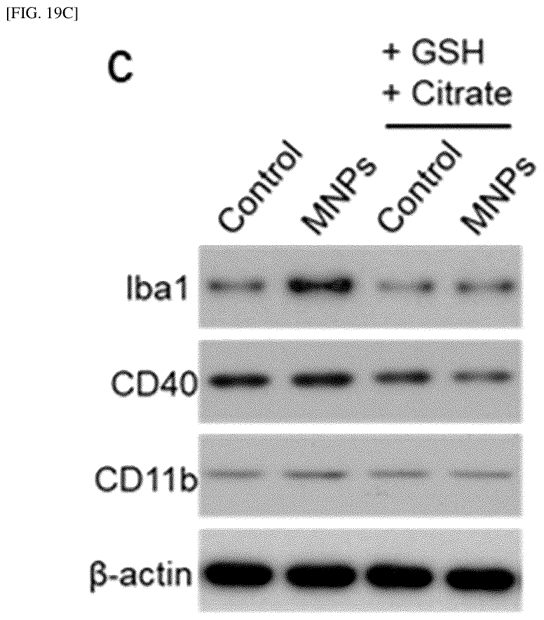

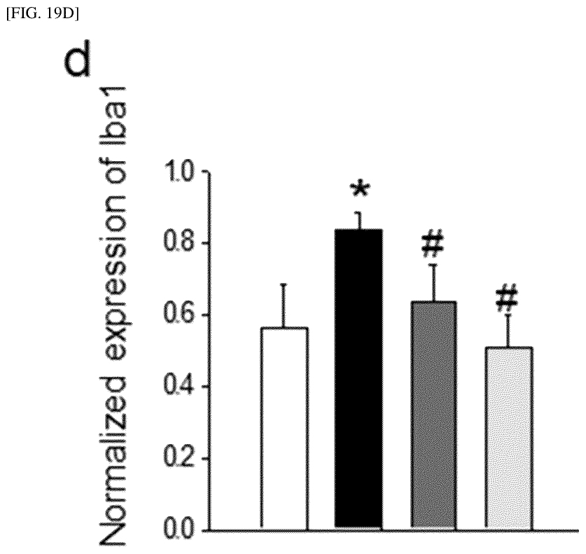

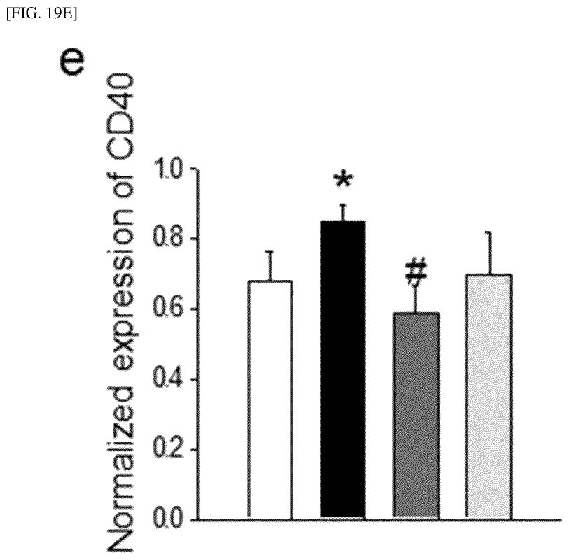

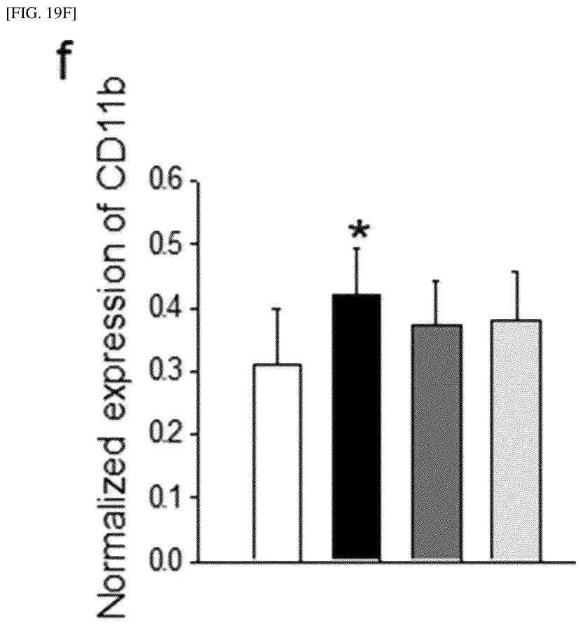

FIG. 19A is a result of immunohistochemical analysis of the morphology of MNPs@SiO₂(RITC) and microglia distributed in the brain cerebellum area of the mice co-administrated with MNPs@SiO₂(RITC) and the mixture of glutathione and citric acid. The decrease in filament length was quantitatively analyzed by treating MNPs@SiO₂(RITC) (FIG. 19B).

Compared to the control group, the filament length of microglia distributed in the brain cerebellum area of the mice treated only with MNPs@SiO₂(RITC) was statistically, significantly decreased, and it was confirmed that such decrease was mitigated in mice co-administrated with the mixture of glutathione and citric acid.

### 2. Immunoblot Analysis

The removed brain tissue was separated into cortex, striatum, hippocampus, thalamus, and cerebellum and dissolved in a solution composed of 20 mM of pH 7.5 Tris-HCl , 150 mM of NaCl, 1 mM of Na2 EDTA, 1 mM of EGTA, 1% NP-40, 1% sodium deoxycholate, 2.5 mM of sodium pyrophosphate, 1 mM of β-glycerophosphate, 1 mM of Na3VO4, and 1 μg/ml of leupeptin. The protein concentration of the dissolved tissue was quantified with a BCA kit (Thermo Fisher Scientific, USA). Samples adjusted to the same concentration were separated by size via polyacrylamide gel electrophoresis (SDS-PAGE), and then proteins were adsorbed onto a nitrocellulose membrane. The protein-containing membrane was blocked with Tris-buffered saline containing 3% skim milk. The blocked membrane was bound with primary antibodies including anti-Iba1, anti-CD40, anti-CD11b, and anti-beta-actin, respectively. After washing the membrane with Tris-buffered saline containing 0.1% Tween-20, the membrane was subjected to the binding with secondary antibodies bound with horseradish peroxidase (HRP) for each primary antibody. After washing with Tris-buffered saline containing 1% Tween-20, the luminescence that appeared by carrying out a reaction with a chemiluminescent solution was printed on an X-ray firm. The size of the protein band appeared thereby was quantified with the Image J program (National Institutes of Health, USA).

FIG. 15D is a result of measuring expression levels of proteins (Iba1, CD40, CD11b) that increase according to the activation of microglia in mouse brain tissues. It was confirmed that the statistical, significant increase of three proteins in the brain hippocampus area of the mice treated only with MNPs@SiO₂(RITC) was inhibited by the mixture of glutathione and citric acid (FIGS. 15E-G).

FIG. 16C is a result of measuring expression levels of proteins (Iba1, CD40, CD11b) that increase according to the activation of microglia in mouse brain tissues. It was confirmed that the statistical, significant increase of three proteins in the brain thalamus area of the mice treated only with MNPs@SiO₂(RITC) was inhibited by the mixture of glutathione and citric acid (FIGS. 16D-F).

FIG. 17C is a result of measuring expression levels of proteins (Iba1, CD40, CD11b) that increase according to the activation of microglia in mouse brain tissues. It was confirmed that the statistical, significant increase of three proteins in the brain cortex area of the mice treated only with MNPs@SiO₂(RITC) was inhibited by the mixture of glutathione and citric acid (FIGS. 17D-F).

FIG. 18C is a result of measuring expression levels of proteins (Iba1, CD40, CD11b) that increase according to the activation of microglia in mouse brain tissues. It was confirmed that the statistical, significant increase of three proteins in the brain striatum area of the mice treated only with MNPs@SiO₂(RITC) was inhibited by the mixture of glutathione and citric acid (FIGS. 18D-F).

FIG. 19C is a result of measuring expression levels of proteins (Iba1, CD40, CD11b) that increase according to the activation of microglia in mouse brain tissues. It was confirmed that the statistical, significant increase of three proteins in the brain cerebellum area of the mice treated only with MNPs@SiO₂(RITC) was inhibited by the mixture of glutathione and citric acid (FIGS. 19D-F).

Although specific parts of the present invention have been described in detail above, it is clear for those skilled in the art that these specific descriptions are merely preferred example embodiments and the scope of the present invention is not limited thereto. Accordingly, the substantial scope of the present invention will be defined by the appended claims and equivalents thereof.

What is claimed is:

1. A method of inhibiting nanotoxicity comprising administering a composition comprising a peptide-based compound, an organic acid, or a mixture thereof as an active ingredient to a subject in need of treating the nanotoxicity.

2. The method of claim 1, wherein the peptide-based compound is glutathione (GSH).

3. The method of claim 1, wherein the organic acid is citric acid.

4. The method of claim 1, wherein the composition comprises the peptide-based compound at a concentration of 0.1 to 0.5 mM.

5. The method of claim 1, wherein the composition comprises the organic acid at a concentration of 0.5 to 2 mM.

6. The method of claim 1, wherein the peptide-based compound and the organic acid are mixed in a concentration ratio of (0.05 to 10):1 in the mixture.

7. The method claim 1, wherein the nanotoxicity is an intracellular toxicity induced by nanoparticles or environmentally-derived particulate matters.

8. The method of claim 1, wherein the composition mitigates intracellular ATP reduction, cell viability reduction, cellular inflammatory morphological changes, and cellular activity induced by nanoparticles or environmentally-derived particulate matters.

9. The method of claim 7, wherein the nanoparticles or environmentally-derived particulate matters are selected from the group consisting of magnetic nanoparticles, inorganic nanoparticles, metal nanoparticles, quantum dot nanoparticles, carbon nanotubes, microplastics, and urban particulate matters.

10. The method of claim 1, wherein the composition inhibits the nanotoxicity in a cell of the subject and the cell is selected from the group consisting of microglia, neurons, astrocytes, and oligodendrocytes.

11. The method of claim 1, wherein the composition is a cosmetic composition.

12. The method of claim 1, wherein the composition is a pharmaceutical composition.

13. The method of claim 1, wherein the composition is a health food composition.
